(19) United States
(12) Patent Application Publication
(10) Pub. No.: US 2021/0020065 A1
(43) Pub. Date: Jan. 21, 2021

(54) TRANSLATOR WITH IMPROVED DATABASE INFORMATION TRANSMISSION

(71) Applicant: Georgetown University, Washington, DC (US)

(72) Inventors: Ophir Frieder, Chevy Chase, MD (US); Joe Garman, Washington, DC (US)

(21) Appl. No.: 16/987,029

(22) Filed: Aug. 6, 2020

Related U.S. Application Data
(63) Continuation of application No. 15/626,006, filed on Jun. 16, 2017, now abandoned.
(60) Provisional application No. 62/351,067, filed on Jun. 16, 2016.

(51) Int. Cl. G09B 19/00; G06F 3/0482; G09B 5/02; G09B 7/00; G16H 50/20; G06F 40/58

(52) U.S. Cl. CPC G09B 19/00; G06F 3/0482; G06F 40/58; G09B 7/00; G16H 50/20; G09B 5/02

(57) ABSTRACT

Technology for providing a translated content item containing text is disclosed. In one example, a method is used for providing a translated content item and a plurality of actions. The method can include generating a base knowledge level of terminology and generating a translated content item having a plurality of translated text, wherein the plurality of translated text is generated based at least in part on the base knowledge level. The method can further include generating a query to verify an understanding of the translated content item and updating the base knowledge level of terminology based on a response to the query.

### CROSS-REFERENCE TO RELATED APPLICATION

This is a Continuation of U.S. patent application Ser. No. 15/626,006, filed Jun. 16, 2017, which claims the benefit of U.S. Provisional Patent Application No. 62/351,067, filed Jun. 16, 2016, both of which are hereby incorporated by reference.

### BACKGROUND

Physicians go through years of schooling and training to diagnose and treat their patients appropriately and have a technical understanding of their field that most likely far exceeds that of their patients. Both medical diagnoses and proposed treatments are likely to contain sophisticated terminology that are foreign to a nonmedical lay person or even a medical practitioner in a different field of practice. The potential for misunderstanding of complex medical/clinical information is increased by an increasing aging population coupled with a shortage of medical practitioners, leading to more patients with less practitioner time per patient. Accordingly, there is a need to ensure patients understand medical terminology that is relevant to their aliment diagnosis and treatment.

### SUMMARY

In an embodiment, a system for text translation is provided. The system includes a data store configured to store a database comprising a plurality of content items each comprising text and a processor. The processor is configured to generate a base knowledge level of terminology and to generate and transmit a translated content item having translated text. The translated content item is based at least in part on a selected one of the plurality of content items, and the translated text is generated based at least in part on the base knowledge level. The processor is further configured to generate and transmit a query/question to verify an understanding of the translated content item and to update the base knowledge level of terminology in the database based on a response to the query received by the processor.

In another embodiment, a method of text translation and action generation is provided. The method includes accessing a profile stored in a database. The profile includes a base knowledge level of terminology. The method also includes determining an understanding of a translated content item having translated text, with the determination being based at least on a response received regarding the translated text. The method further includes updating the base knowledge of terminology in the profile based at least on the response received regarding the translated text and determining a plurality of actions/actionable options for a diagnosis based on the translated content item.

In yet another embodiment, a non-transitory computer readable storage medium is provided for storing computer-executable instructions that when executed perform a method of text translation. The method includes receiving, by a server, a content item comprising text, and generating and transmitting a translated content item by the server. The translated content item includes translated text in the content item. The method also includes verifying an understanding of the translated content item, and generating and transmitting a plurality of actions based on a diagnosis of a condition identified from the content item.

As further described below, an intelligent medical hypertext translator can provide enormous utility to patients having varying levels of medical skill and knowledge, and who may additionally have only limited time with a health care practitioner. The ability to translate complex medical terms into more understandable language can provide a patient with a better understanding of a prognosis, the severity of the prognosis, and additional steps that could be taken to alleviate or lessen the severity of a given prognosis.

Additionally, an intelligent medical hypertext translator can provide knowledge, practices, and other information stored in a database that the medical practitioner may have simply forgotten to mention. This could include information that is especially pertinent to a patient based on sex, age, race, etc.

Further, an intelligent medical hypertext translator that can generate questions to a patient to assess their understanding of a prognosis, and even scale the lexicographical degree of translation, can provide an assurance that patients with differing levels of understanding of medical terms and recommendations are accommodated for.

An intelligent medical hypertext translator can have further advantages in terms of computing efficiency and customer flexibility. For example, the disclosed translator can be capable of machine learning. This can speed processing time based on responses to one or more questions generated by the system. In the case of a commonly misunderstood term, such as “acute viral rhinopharyngitis,” the system may initially tell a patient/user that they have “a cold.” However, upon learning that many people understand this condition to be something more similar such as “sick,” the system may change its response from “cold” to “sick,” while still providing all of the necessary health information and recommendations that come along with having a cold. Such learning can speed up processing by prioritizing commonly used terms. Such a system would also be capable of re-prioritizing commonly used terms (for example, “peepers,” an antiquated term for eyes, is simply re-prioritized to “eyes”). In doing so, a user needn't search and re-search for terms that do not make sense to them, saving processing resources.

A further advantage of such a system could be in speech-to-text translation. One advantage of this approach is that people use natural language in a way that is very different from how they type messages/queries. By employing speech-to-text, one or more advantages can be achieved. For one, the system can be programmed (by users) to more accurately reflect how a non-medically trained users think about their bodies and conditions, which can again save processing time based on a computed probability that the patient will understand the translation. Secondly, users who may be illiterate, or suffering from conditions such as carpel tunnel syndrome that make text entry difficult are accommodated.

Further, server-based embodiments of the intelligent medical hypertext translator as described herein provide numerous internet-centric and/or technical advantages. For example, by implementing the medical hypertext translator in one or more servers (e.g., cloud-based servers) that communicate with one or more user devices via a network (e.g., the internet), the storage device overhead and memory overhead at the user's device can be substantially reduced by avoiding the need to store a local copy of the translator. Similarly, computational resources at the user's device are saved in by this arrangement by offloading the execution of the disclosed methods to a remote server. Still further, the server-based intelligent medical hypertext translator allows updates to the data store to be made in a computationally efficient manner by avoiding the need to push out updates to all user devices having the translator, which can be unreliable and costly in terms of computational and network resources. Thus, embodiments of the disclosed technology improve the function and performance of certain computing devices. Still further, the web- and server-based embodiments can provide access to the disclosed methods to computing devices that operate using different platforms (e.g., operating systems) and/or have different performance capabilities, thus allowing the disclosed methods to be performed for a larger set of devices than would otherwise be possible. Additionally, certain embodiments access third party website content source, thus providing an internet-centric knowledge base that has no pre-internet analogue.

An exemplary embodiment of a system in accordance with the present disclosure can include a data store comprising a plurality of content items, the plurality of content items each comprising a plurality of medical terms, and a processor. The processor can be configured to generate a base user knowledge level of medical terminology for a user, generate a translated content item having a plurality of translated medical terms, wherein the translated content item is based at least in part on a select one of the plurality of content items and wherein the plurality of translated medical terms are generated based at least in part on the base user knowledge level, and generate a question to verify user understanding of the translated content item, and update the base user knowledge level of medical terminology based on a response to the question received by the processor. In some embodiments, generating the base user knowledge level of medical terminology can include providing the user with a vocabulary test.

In some embodiments, the processor can be further configured to generate a plurality of actionable options, wherein the plurality of actionable options are based at least in part on the select one of the plurality of content items. Additionally and/or alternatively, generating the plurality of actionable options can compromise determining a diagnosis of a medical condition, wherein the diagnosis can be based at least in part on the select one of the plurality of content items. Additionally and/or alternatively, generating the plurality of actionable options can compromise ranking the plurality of actionable options based on one or more of relevance, importance to treatment of a medical condition, potential benefit versus harm.

In some embodiments, the processor can be further configured to generate one or more questions to verify user understanding of the plurality of actionable options. Additionally and/or alternatively, the processor can be further configured to receive a user selection of at least one of the plurality of actionable options and generate a treatment plan based at least in part on the user selection. In some embodiments, generating the translated content item can compromise identifying a plurality of potentially misunderstood medical terms and providing a translated term for each of the plurality of potentially misunderstood medical terms.

An exemplary embodiment of a method in accordance with the present disclosure can include identifying a user based at least in part on a stored user profile, wherein the user profile comprises a user base knowledge of medical terms, determining user understanding of a translated content item having translated medical terms based at least on a response received from the user regarding at least one of the translated medical terms, updating the user base knowledge of medical terms in the stored user profile based at least on the response received from the user regarding the translated medical terms, and determining a plurality of actionable options for a medical diagnosis based on the translated content item. In some embodiments, determining user understanding of the translated content item can compromise generating a question based on one or more of the translated medical terms. This approach can be beneficial in that the computing efficiency of a server that has identified the customer can perform more optimal suggestions, thus enhancing processing speed and reducing time to create matches based on the user's unique responses to one or more questions generated by the system.

Additionally and/or alternatively, the method can further compromise translating a plurality of potentially misunderstood medical terms in the plurality of actionable options. Additionally and/or alternatively, the method can further compromise determining user understanding of the plurality of actionable options based at least on a response received from the user based at least in part on a translated medical term in the plurality of actionable options. Additionally and/or alternatively, the method can further compromise updating the user base knowledge of medical terms in the stored user profile based at least on the response received from the user regarding the translated medical term in the plurality of actionable options. Additionally and/or alternatively, the method can further compromise generating a treatment plan based on the plurality of actionable options. In some embodiments, generating the treatment plan can compromise providing the plurality of actionable options to the user and receiving user selection of one or more of the plurality of actionable options. This form of “machine learning” can increase computing efficiency because different forms of terminology for human anatomy, illness, and treatment can be adapted on a per-patient basis and used subsequently on a compartmentalized scale (for example, elderly patients may have different terms for illnesses or anatomy than younger patients). By learning and compartmentalizing such differences on a per-patient basis, computational resources can be minimized.

An exemplary embodiment of computer readable storage medium storing computer-executable instructions that when executed perform a method in accordance with the present disclosure can include the method comprising receiving a content item having a plurality of potentially misunderstood medical terms, generating a translated content item, wherein the translated content item provides a translation for each of the plurality of potentially misunderstood medical terms in the content item, verifying user understanding of the translated content item, and generating a plurality of actionable options based on a diagnosis of a medical condition identified from the content item. In some embodiments the computer readable storage medium of can further comprise verifying user understanding of the plurality of actionable options.

The foregoing and other objects, features, and advantages of the invention will become more apparent from the following detailed description, which proceeds with reference to the accompanying figures.

### BRIEF DESCRIPTION OF THE DRAWINGS

FIG. 1 is a system diagram showing an example architecture for intelligent medical hypertext information translator of content.

FIG. 2 is a flow diagram illustrating an exemplary process of translating or interpreting a medical text for a user.

FIG. 3 is an exemplary illustration of an example medical report.

FIG. 4 is an exemplary illustration of the example medical report of FIG. 3 in which a plurality of potentially misunderstood terms are highlighted.

FIG. 5 is an exemplary illustration of a list of the plurality of potentially misunderstood terms from the example medical report of FIG. 4.

FIG. 6 is an exemplary illustration of a list of the plurality of potentially misunderstood terms of FIG. 5, along with corresponding translations or interpretations of the plurality of potentially misunderstood terms.

FIG. 7 is a flow diagram illustrating an exemplary process of verification of user understanding of a medical text.

FIG. 8 is a flow diagram illustrating an exemplary process of acquisition and provision of a plurality of actionable options.

FIG. 9 is an exemplary illustration of a plurality of actionable options.

FIG. 10 is a flow diagram illustrating an exemplary process of verification of user understanding of a plurality of actionable options and generation of a treatment plan.

FIG. 11 is a flow diagram illustrating an exemplary process of testing a user's potential understanding of a plurality of actionable options.

FIG. 12 is a flow diagram illustrating an exemplary method of translating a content item for a user.

FIG. 13 depicts a generalized example of a suitable computing environment in which the described innovations may be implemented.

### DETAILED DESCRIPTION

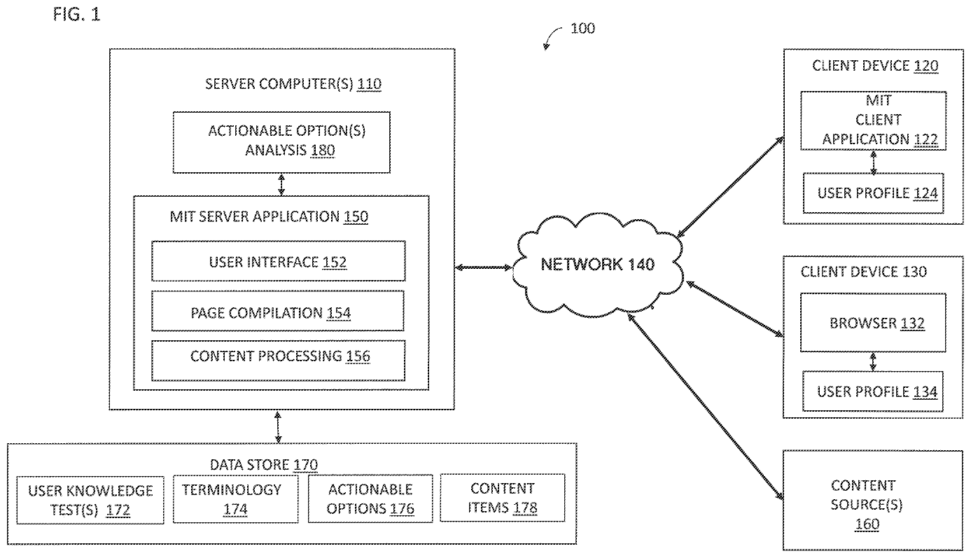

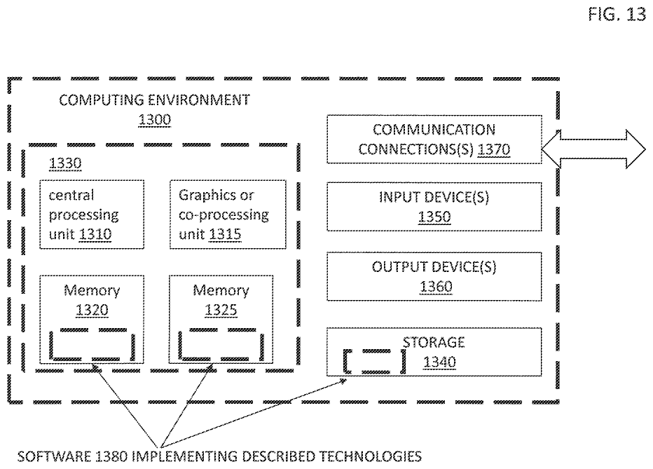

FIG. 1 is a system diagram showing an example architecture 100 for a medical information translator (“MIT”) system. The architecture 100 can be a client-server architecture including one or more server computer(s) 110 and one or more client devices 120 and 130 communicating over a network 140. The one or more server computer(s) 110 can include a computing environment as described further below, with reference to FIG. 13. The network 140 can include a local area network (LAN), a Wide Area Network (WAN), the Internet, an intranet, a wired network, a wireless network, a cellular network, combinations thereof, or any network suitable for providing a channel for communication between the server computer(s) 110 and the client devices 120 and 130. It should be appreciated that the network topology illustrated in FIG. 1 has been simplified and that multiple networks and networking devices can be utilized to interconnect the various computing systems disclosed herein. The client devices 120 and 130 can be a mobile device, a desktop computer, a game console, a set-top box, a laptop computer, a tablet computer, a personal digital assistant, a smartphone, a cellular phone, or any suitable computing environment, for example.

A MIT software application can be a service or a platform for enabling assessment of user knowledge level, analysis and interpretation of medical information at the appropriate user knowledge level, verification of user understanding of the interpreted medical information, provision of actionable options, and/or verification of user understanding of the actionable options. Assessing a user's level of knowledge can enable the MIT software application to save computing resources by honing in on a user's level of knowledge and establishing a theoretical baseline of information that the system deduces the patient can understand, saving computing resources and expediting the speed at which a translation may be provided.

As one example, the MIT software application can include a MIT server application 150 executing on the server computer(s) 110 and a MIT client application 122 executing on the client device 120. As another example, the MIT software application can include a MIT server application 150 executing on the server computer(s) 110 that is accessible using a browser 132 executing on the client device 130. The MIT client application 122 and/or the MIT server application 150 can include various software modules, such as a user interface module 152, a user profile module, a page compilation module 154 and a content processing module 156. The MIT software application can present content items and controls to a user using pages that can be displayed on the client devices 120 and 130. For example, the controls can include buttons or menus to add content items, share content items, comment on content items, respond to questions regarding content items, and/or respond to a user knowledge test.

As previously discussed, server-based embodiments can provide internet-centric and/or technical advantages, for example, by implementing functionality in one or more servers (e.g., cloud-based servers) that communicate with one or more user devices via a network (e.g., the internet). In doing so, the storage device overhead and memory overhead on the user's device can be substantially reduced by avoiding the need to store a local copy of the translator. Similarly, computational resources at the user's device are saved in by this arrangement by offloading the execution of the disclosed methods to the remote server. Still further, the server-based translator allows updates to the data store to be made in a computationally efficient manner by avoiding the need to push out updates to all user devices having the translator, which can be unreliable and costly in terms of computational and network resources. The web- and server-based models have a further advantage of providing access to computing devices that operate using different platforms and/or have different performance capabilities.

The user interface module 152 can be used to communicate with users and to identify and authenticate the users. Specifically, the user interface module 152 can receive requests for pages and requests to perform actions associated with the pages. The user interface module 152 can respond to the requests for the pages and can perform actions associated with the pages. For example, a user can begin using the MIT software application by navigating to a start page and/or by logging into the MIT software application. As a specific example, a user using the client device 130 can navigate to the start page by selecting a uniform resource locator (URL) corresponding to the start page. The URL can be selected by starting the browser 132 (such as when the start page is a home page of the browser) or by interacting with a user interface (UI) of the browser 132, such as by clicking on a hyperlink, entering the URL in an address bar, or selecting the URL from a favorites list. When the user selects the URL of the start page, a request for the start page can be sent to the user interface module 152. The user interface module 152 can transmit the start page to the client device 130 and the start page can be displayed in a window of the browser 132. The start page can provide a prompt for the user to enter credentials, which can be transmitted to the user interface module 152. If the received credentials match stored credentials of a user of the community of users, the user can begin using the MIT software application. As another example, the user credentials can be stored in a user profile 134 (such as a cookie) and authentication can occur without prompting the user to enter his or her credentials. As another example, a start page can be displayed on the client device 120 when the MIT client application 122 is started on the client device 120. In particular, the MIT client application 122 can transmit a request for the start page and credentials from the user profile 124 and the user interface module 152 can transmit the start page to the MIT client application 122 for display if the user is authorized.

In addition to providing credentials for a user, the user profile 134 can be used to identify the user when the user creates a content item and/or interacts with a content item generated by other users. Additionally and/or alternatively, the user profile 134 can be used to store a user's medical terminology knowledge level as assessed by the MIT software. Additionally and/or alternatively, the user profile 134 can be used to store a treatment plan comprising one or more actionable options.

The page compilation module 154 can assemble and format pages for viewing by the user. For example, the pages can be web pages or other types of pages describing information to be displayed in a graphical user interface. The pages can include content items, functional items (such as controls) for interacting with and/or creating the content items, user knowledge tests, controls for responding to user knowledge tests, and other information. For example, the page compilation module 154 can retrieve all content items for a given page, insert any controls and/or status information for the page, and format the page for display. The pages can be described using one or more of various languages, such as HyperText Markup Language (HTML), Cascading Style Sheets (CSS), JavaScript, Extensible Markup Language (XML), or other suitable languages for describing interactive pages. The page compilation module 154 can retrieve content for the pages from a data store 170 and/or the content processing module 156.

The content processing module 156 can be used for analyzing content items, identifying medical terminology and translating the identified terminology in accordance with a user's measured literacy level. The content processing module 156 can retrieve appropriately translated medical terminology from the data store 170. For example, a new content item provided by a user can be analyzed to determine which of the medical terms are likely to be potentially misunderstood by the user. Once a plurality of potentially misunderstood medical terms are identified, the content process module 156 can supplement the content item with translations of the plurality of potentially misunderstood medical terms. The translations may be based at least in part on a knowledge level of the user. The content processing module 156 can retrieve content from the content sources 160, client devices 120, 130, data store 170 and/or the actionable options analysis 180.

The content processing module 156 can further select subject material from content item having translated medical terms to test a user's understanding of the content item with translated medical terms. The content processing module 156 can generate a question from the selected subject material and determine if a response is correct. If the content processing module 156 determines that the user does not understand the content item, an option to contact a medical professional can be provided.

The data store 170 can include computer readable storage used for storing the user knowledge test(s) 172, terminology 174, actionable options 176 and content items 178. The data store 170 can include removable or non-removable storage devices, including magnetic disks, direct-attached storage, network-attached storage (NAS), storage area networks (SAN), redundant arrays of independent disks (RAID), magnetic tapes or cassettes, CD-ROMs, DVDs, or any other medium which can be used to store information in a non-transitory way and which can be accessed by the server computer(s) 110. The data store 170 can include a relational database. A relational database can include a number of tables that each has one or more columns and one or more rows. The user knowledge test(s) 172 can include means to assess a user's knowledge and comprehension levels, such as text, documents, graphics, photos, videos, and audio recordings. The terminology 174 can include a compilation of medical terminology, each term translated to meet various levels of medical terminology literacy. The actionable options 176 can include treatment options for various health conditions. The content items 178 can include content items generated or provided by a user and content items generated or provided by other sources, such as content source(s) 160 and/or actionable options analysis 180. The content item may comprise one or more of text, documents, graphics, photos, videos, and/or audio recordings comprising medical terminology. Examples of a content item include, but are not limited to, a medical report from a hospital or physician's office, text from a medical journal, dictation from a medical professional, and/or a plurality of actionable options generated by the actionable options analysis 180. Content source(s) 160 can include physician offices, third party websites and/or other sources of text containing medical terminology, and/or the actionable options analysis 180.

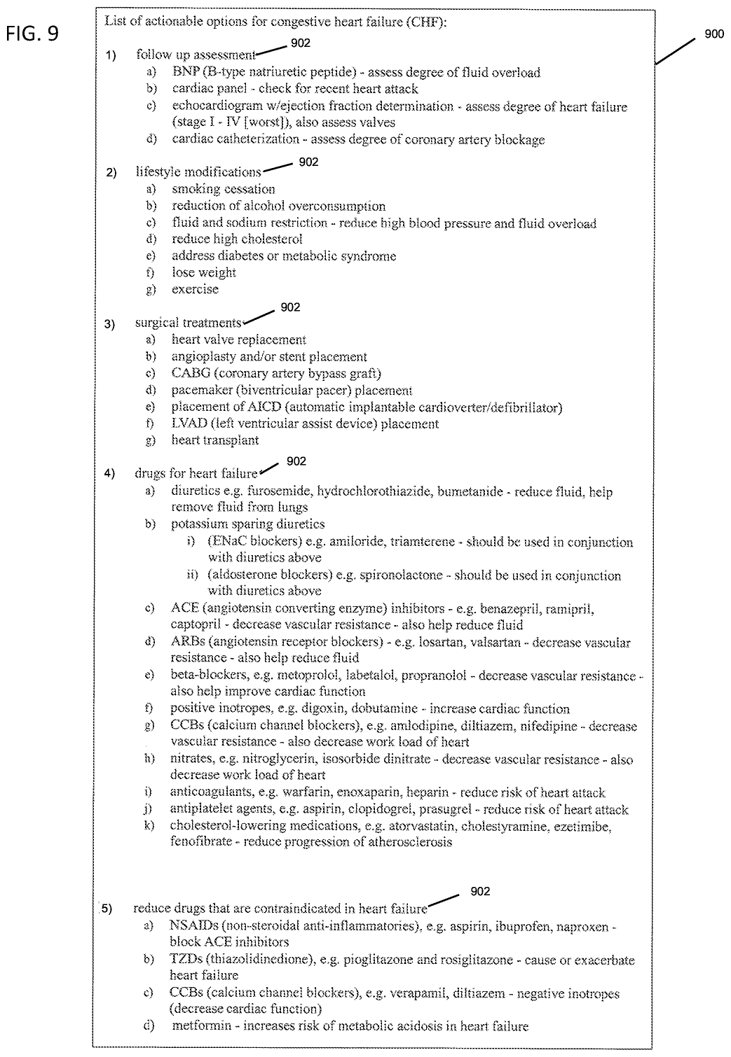

The actionable option(s) analysis module 180 can be a software module used to analyze a content item of the MIT server application 150, such as a medical report, to identify patient problems, e.g. diagnosis, and determine appropriate treatment options, also referred to as actionable options. The actionable option(s) analysis module 180 can execute on the server computer(s) 110 (as illustrated) or on the client devices 120 and 130. As an example, the actionable option(s) analysis module 180 can interface with or be incorporated into the MIT server application 150 so that the content items can be analyzed in view of the actionable options 176. In particular, the actionable option(s) analysis module 180 can provide a plurality of actionable options (such as page 900 of FIG. 9). The plurality of actionable options can be ranked, for example by relevance, importance, and weight of beneficial outcome. This ranking can consider the indication, contra-indication, benefit, and/or harm of each actionable option. The plurality of actionable options can be translated prior to presentation to a user in accordance with the user's knowledge level by the content processing module 156. Additionally and/or alternatively, a user's understanding of the plurality of actionable options can be tested by the content processing module 156, as described above.

Additionally and/or alternatively, the plurality of actionable options can be presented to a user one-at-a-time with provision of benefits and drawbacks of each. If any additional information is needed to choose or reject the actionable option, then the option to pursue additional information can be provided. The user can be provided with control to select or reject one or more of the plurality of actionable options. The plurality of actionable options selected by the user can be used to generate a treatment plan for the user. Alternatively, the plurality of actionable options can be used as a treatment plan for the user.

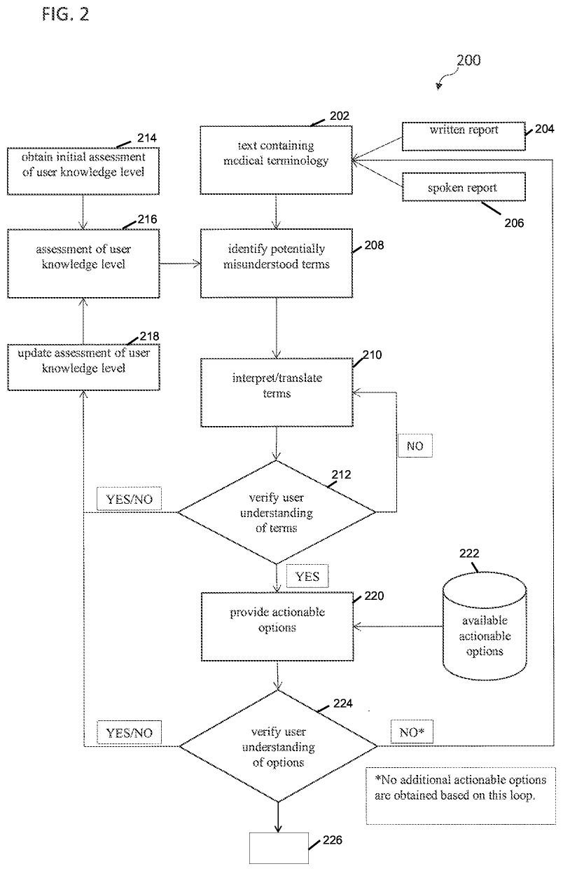

FIG. 2 is a flow diagram illustrating an exemplary method 200 for translating or interpreting a medical text for a user. At 202, a content item such as a written medical report 204 or a spoken medical report 206 can be provided. At 208, the content item can be analyzed to identify a plurality of potentially misunderstood terms. The plurality of potentially misunderstood terms can be identified based at least in part on a user's measured medical terminology literacy level. For example, at 214 an initial assessment of a user's medical terminology knowledge level can be obtained. At 216, the user knowledge level can be assessed and can be provided at 208 to aid in identification of the plurality of misunderstood terms. Alternatively, the plurality of potentially misunderstood terms may be the same for each user, regardless of medical terminology literacy level. At 210, the plurality of potentially misunderstood terms can be interpreted or translated. For example, the plurality of potentially misunderstood terms can be construed based on at least in part on the user's measured medical terminology knowledge level. Alternatively, the plurality of potentially misunderstood terms may be construed the same for each user, regardless of medical terminology knowledge level.

At 212, the user's understanding of a term can be verified. The verification or failure to verify understanding of the translation of the plurality of potentially misunderstood terms can be provided at 218 and can be used to update the assessment of user knowledge level at 216. If the user does not understand the translation of the plurality of potentially misunderstood terms, the process can return to 210 for translation. If the user does understand the translation of the plurality of potentially misunderstood terms, at 220 a plurality of actionable options can be provided to the user. The plurality of actionable options can be provisioned from a database of available actionable options 222.

At 224, the user's understanding of the plurality of actionable options can be verified. The verification or lack thereof can be used to update the assessment of the user's knowledge level at 218. If the user does not understand the plurality of actionable options, the plurality of actionable options may be provided at 202 as a content item text containing medical terminology. This process may continue until user understanding is verified. At 226, the process ends.

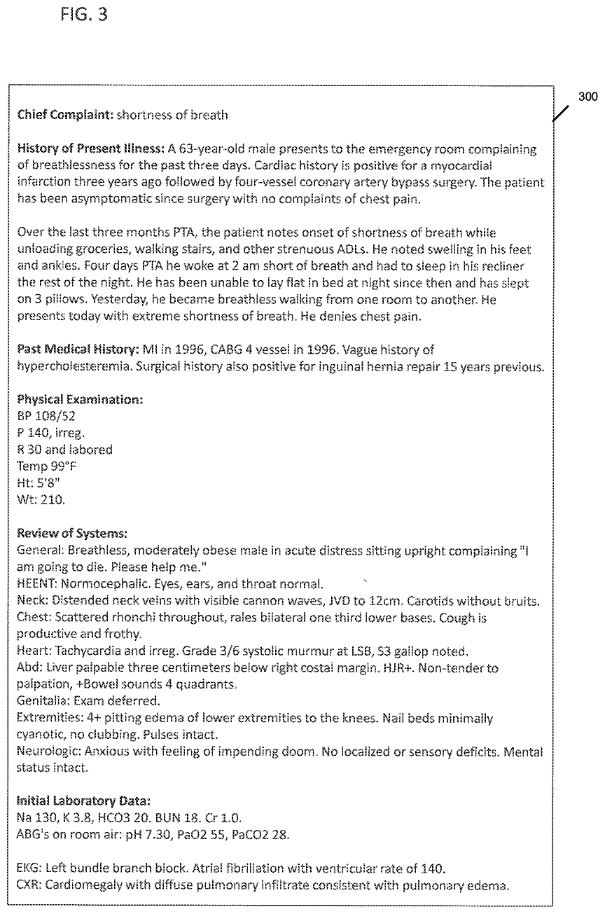

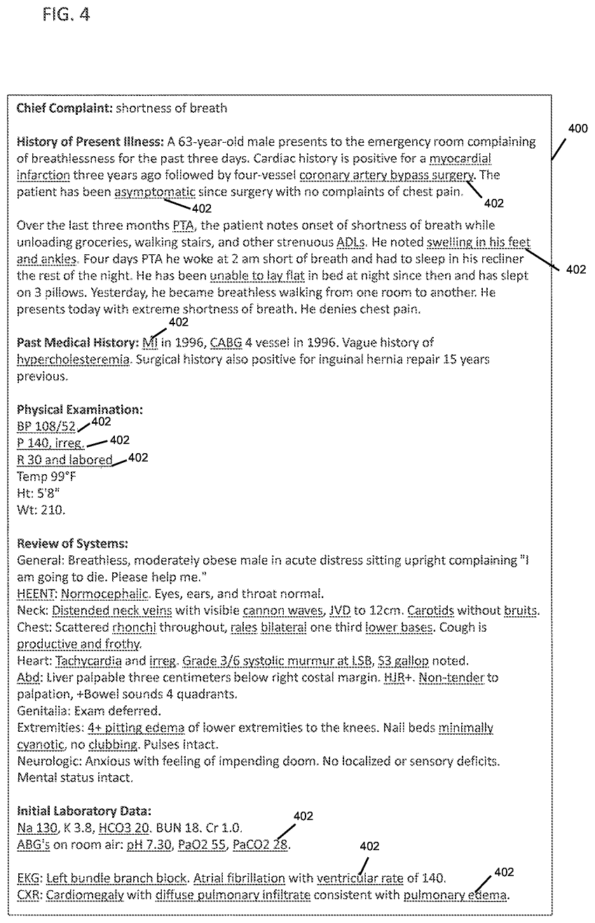

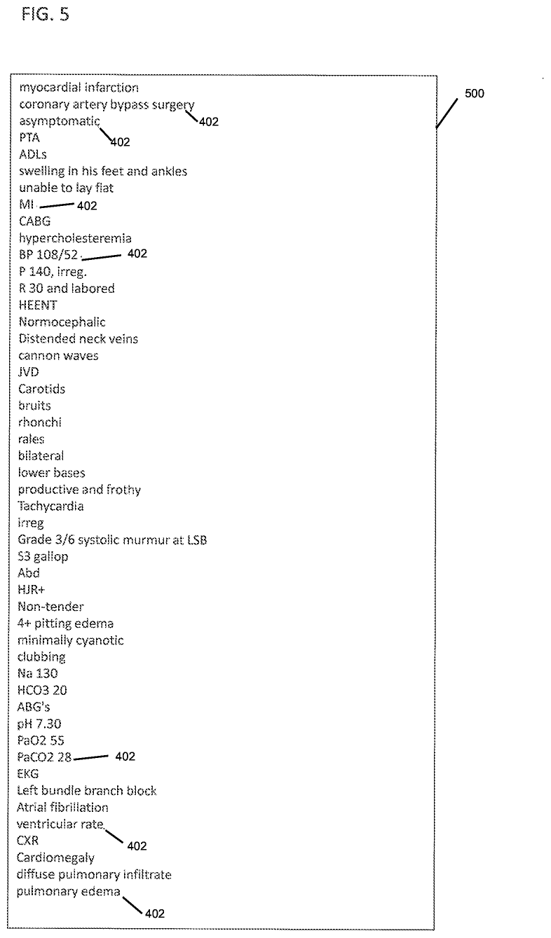

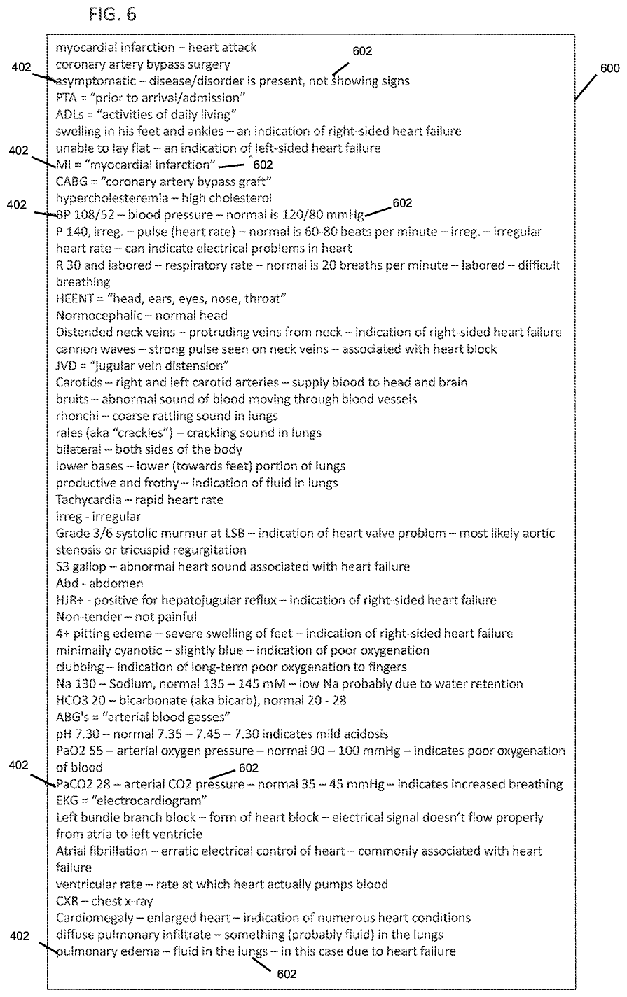

FIG. 3 illustrates an exemplary page of a content item, for example a medical report 300. The medical report may comprise a plurality of medical terms. FIG. 4 is an exemplary illustration of an example medical report 400 in which a plurality of potentially misunderstood terms 402 are identified and shown as underlined. FIG. 5 is an exemplary illustration of a term list 500 of the plurality of potentially misunderstood terms 402 extracted from the example medical report 400 of FIG. 4. FIG. 6 is an exemplary illustration of a translation list 600 of the plurality of potentially misunderstood terms 402 along with corresponding translations or interpretations 602 of the plurality of potentially misunderstood terms. The corresponding translations or interpretations 602 of the plurality of potentially misunderstood terms 402 can be based at least in part on an assessment of a user's medical knowledge level.

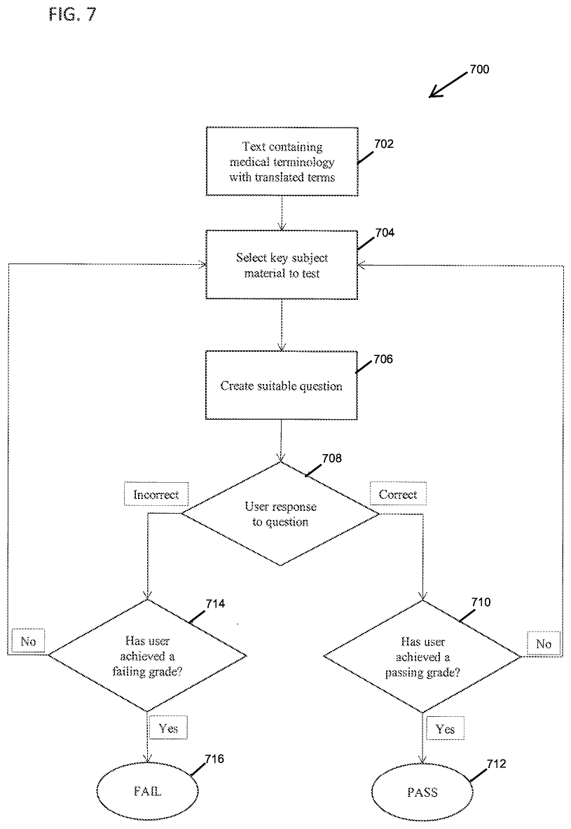

FIG. 7 illustrates an example method 700 of verification of user understanding of a content item. At 702, a medical text comprising a first plurality of translations of a plurality of potentially misunderstood terms can be provided. At 704, subject material to test a user can be selected from the medical text. In some embodiments, the selected subject material can be considered key or critical to the user, for example pharmaceutical drug interactions, allergens, etc. At 706, a question can be created based at least in part on the selected subject matter.

At 708, determination can be made whether a user response to the question was correct or incorrect. At 710, if the user response is determined to be correct, a determination can be made whether the user comprehends or understands the medical text. For example, the determination can be made based at least in part on the number of questions correctly answered. Additionally and/or alternatively, the determination can be made based at least in part on the critical nature of question(s) answered correctly or incorrectly. If the determination is yes, then at 712 the process can be ended. If the determination is no, the process may return to 704 for further testing.

At 714, if the user response is determined to be incorrect, a determination can be made whether the user comprehends or understands the medical text. For example, the determination can be made based at least in part on the number of questions correctly answered. Additionally and/or alternatively, the determination can be made based at least in part on the critical nature of question(s) answered correctly or incorrectly. If the determination at 714 is no, the process may return to 704 for further testing. If the determination at 714 is yes, then at 716 the process 700 can be ended. Additionally and/or alternatively, in some embodiments, if at 716, the user has achieved a failing grade, then a medical professional may be informed to contact the user and/or the user may be referred to a medical professional. Additionally and/or alternatively, if the user achieves a failing grade at 716, a medical text comprising a second plurality of translations of a plurality of potentially misunderstood terms can be provided at 702 and the process 700 can start again. The second plurality of translations can be simpler or at a reduced complexity then the first plurality of translations and/or may comprise a larger number of translations.

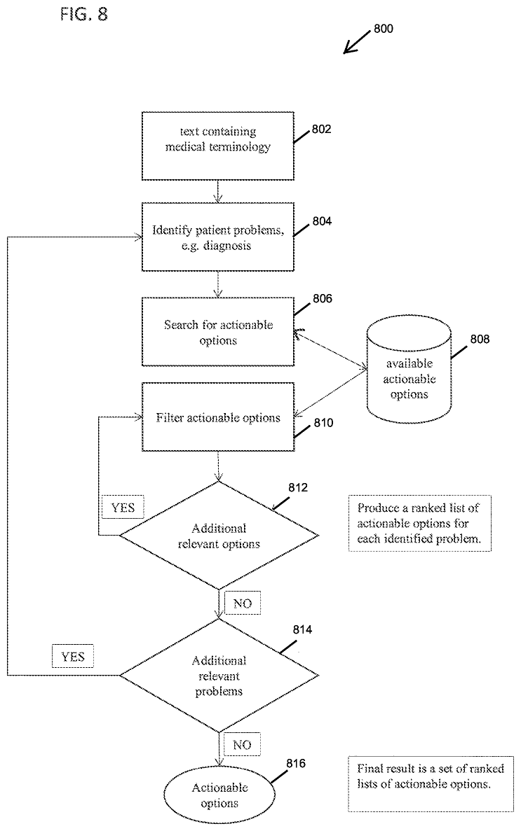

FIG. 8 is a flow diagram illustrating an exemplary method 800 of acquisition and provision of a plurality of actionable options based at least in part on a content item. For example, at 802 a content item, for example a text containing medical terminology can be provided. At 804, one or more patient problem(s) can be identified based at least in part on the text containing medical terminology. At 806, a search for actionable options based at least in part on the one or more patient problems can be performed. One or more available actionable options can be provided by an available actionable options database 808.

At 810, the one or more available actionable options can be filtered. One or more available actionable options can be provided by an available actionable options database 808. At 812, a determination can be made whether there are one or more additional relevant actionable options. If the determination is yes, then the process can return to 810. If the determination is no, at 814 a determination can be made whether there are additional relevant one or more patient problems. If yes, then the process can return to 804. If the determination is no, at 816 the process can end. At 816, a ranked list of actionable options for each of the one or more patient problem(s) can be produced.

FIG. 9 is an exemplary report 900 of a plurality of actionable options 902. The plurality of actionable options 902 can be ranked. For example, the plurality of actionable options 902 can be ranked in order of relevance, importance to treatment of a medical condition, potential benefit versus harm, ease of action, cost, etc. The plurality of actionable options 902 can be presented to a user one at a time or in a single presentation.

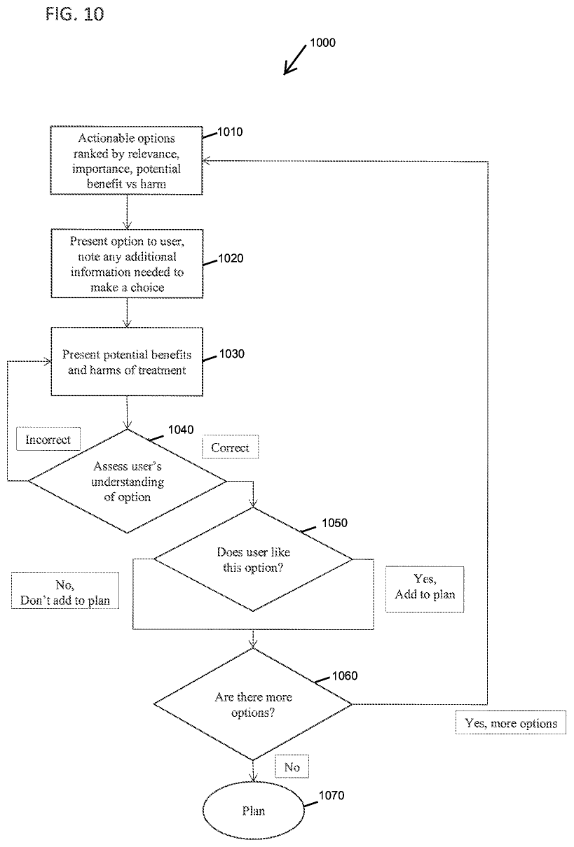

FIG. 10 is a flow diagram illustrating an exemplary method 1000 of verification of user understanding of a plurality of actionable options and generation of a treatment plan. At 1010, a report of a plurality of actionable options can be provided. The plurality of actionable options can be ranked by one or more of relevance, importance, and/or potential benefit to the patient versus potential harm to the patient. At 1020, one of the plurality of actionable options can be presented to a user. A plurality of potentially misunderstood terms can be identified and translated in the one of the plurality of actionable options presented to the user. Additional information regarding the one of the plurality of action options can be identified in the presentation to the user, such as additional information needed to make a choice. At 1030, potential benefits and potential harms of the one of the plurality of actionable options can be presented to the user.

At 1040, a determination of the user's understanding of the one of the plurality of actionable options can be made. For example, the user can be tested regarding the meaning of one or more medical terms used in the one of the plurality of actionable options. If it is determined the user is incorrect, the process can return to 1030. If it is determined the user is correct, a determination can be made at 1050 regarding whether the user prefers the one of the plurality of actionable options presented. If no, the one of the plurality of actionable options may not be added to a treatment plan. If yes, the one of the plurality of actionable options can be added to the treatment plan. At 1060, a determination is made as to if there are more of the plurality of actionable options to present to the user. If yes, then the process 1000 can return to 1010. If no, at 1070 the process can end and/or a treatment plan can be generated.

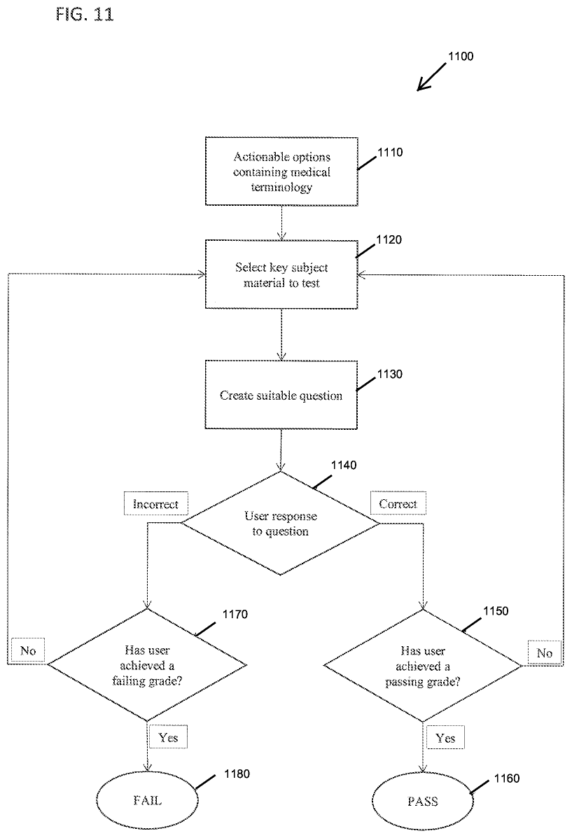

FIG. 11 is a flow diagram illustrating an exemplary method 1100 of testing a user's potential understanding of a plurality of actionable options. At 1110, a plurality of actionable options comprising a first plurality of translations of a plurality of potentially misunderstood terms is provided. At 1120, subject material to test a user can be selected from the plurality of actionable options. In some embodiments, the selected subject material can be considered key or critical to the user, for example pharmaceutical drug interactions, allergens, etc. At 1130, a question can be created based at least in part on the selected subject matter.

At 1140, determination can be made whether a user response to the question was correct or incorrect. At 1150, if the user response is determined to be correct, a determination can be made whether the user comprehends or understands the plurality of actionable options. For example, the determination can be made based at least in part on the number of questions correctly answered. Additionally and/or alternatively, the determination can be made based at least in part on the critical nature of question(s) answered correctly or incorrectly. If the determination is yes, then at 1160 the process can be ended. If the determination is no, the process may return to 1120 for further testing.

At 1170, if the user response is determined to be incorrect, a determination can be made whether the user comprehends or understands the plurality of actionable options. For example, the determination can be made based at least in part on the number of questions correctly answered. Additionally and/or alternatively, the determination can be made based at least in part on the critical nature of question(s) answered correctly or incorrectly. If the determination is yes, then at 1180 the process can be ended. Additionally and/or alternatively, if at 1180, the user has achieved a failing grade, then in some embodiments, a medical professional may be informed to contact the user and/or the user may be referred to a medical professional. Additionally and/or alternatively, if the user achieves a failing grade at 1180, a plurality of actionable options comprising a second plurality of translations of a plurality of potentially misunderstood terms can be provided at 1110 and the process 1100 can start again. The second plurality of translations can be simpler or at a reduced complexity then the first plurality of translations and/or may comprise a larger number of translations.

If the determination at 1170 is no, the process 1100 may return to 1120 for further testing. In some embodiments of the method 1100, the plurality of actionable options may be presented to a user one at a time and the user can be tested regarding comprehension one actionable option at a time.

Some embodiments of a system may comprise a processer programmed to assess the initial user knowledge level, obtain text containing medical terminology, identify potentially misunderstood terms, interpret/translate said terms, verify user understanding of translated terms, re-translate said terms if understanding is not adequate, and provide actionable options if available. In some embodiments, one or more of a questionnaire, a vocabulary test, and/or a reading test can be used in the initial assessment. In some embodiments, the user's anxiety level concerning one or more medical conditions can be assessed. In some embodiments, the user's goal in understanding medical terminology can be assessed.

In some embodiments, the text is obtained from a speech to text input device and/or a medical report. As previously discussed, an advantage of this approach is that transforming speech into text encapsulates peoples natural use of language, which can be very different from how they type messages/queries. By employing speech-to-text, the system can be programmed (by users) to more accurately reflect how a non-medically trained users think about their bodies and conditions, which can save processing time based on a computed probability that the patient will understand the translation. Secondly, users who may be illiterate, or suffering from conditions such as carpel tunnel syndrome that make text entry difficult are accommodated.

In some embodiments, the user can select terms from the text for translation via a pointing device, e.g. mouse or finger, and/or via a spoken request. The user assessment can be updated based on selection of terms and/or based on verification of understanding. In some embodiments, external sources are searched for actionable options. In some embodiments, user understanding of the actionable options is assessed via one or more quizzes.

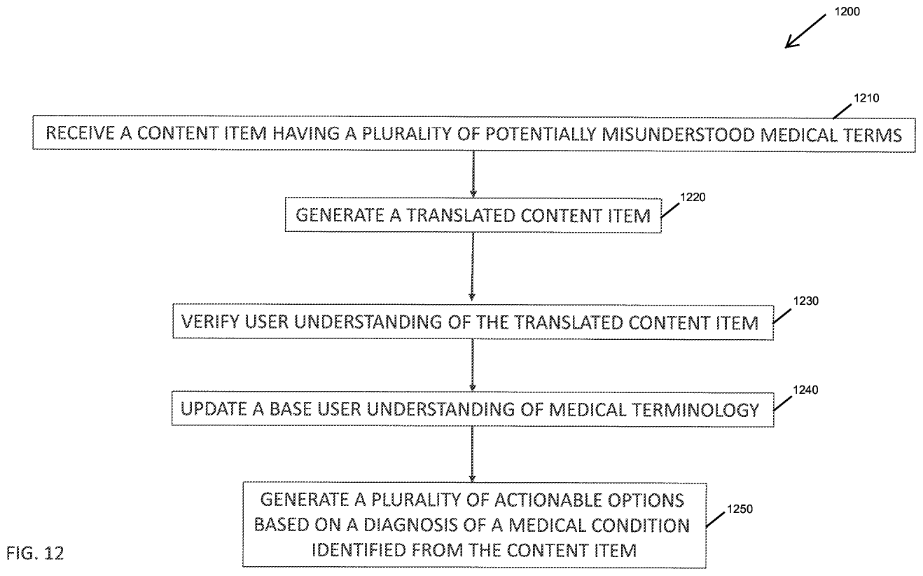

FIG. 12 is a flow diagram of an example of a method 1200 for providing a translated content item for a user. At 1210, a content item having a plurality of potentially misunderstood medical terms can be received. At 1220, a translated content item can be generated. The translated content item can include a translation for each of the plurality of potentially misunderstood medical terms. At 1230, user understanding of the translated content item can be verified. For example, a question based on one or more of the translated terms can be generated. At 1240, a base user understanding of medical terminology can be updated, for example in view of the user understanding or lack thereof of the translated content item and/or response to the question. At 1250, a plurality of actionable options based on a diagnosis of a medical condition identified from the content item can be generated.

FIG. 13 depicts a generalized example of a suitable computing environment 1300 in which the described innovations may be implemented. The computing environment 1300 is not intended to suggest any limitation as to scope of use or functionality, as the innovations may be implemented in diverse general-purpose or special-purpose computing systems. For example, the computing environment 1300 can be any of a variety of computing devices (e.g., desktop computer, laptop computer, server computer, tablet computer, etc.).

With reference to FIG. 13, the computing environment 1300 includes one or more processing units 1310, 1315 and memory 1320, 1325. In FIG. 13, this basic configuration 1330 is included within a dashed line. The processing units 1310, 1315 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 13 shows a central processing unit 1310 as well as a graphics processing unit or co-processing unit 1315. The tangible memory 1320, 1325 may be volatile memory (e.g., registers, cache, RAM), non-volatile memory (e.g., ROM, EEPROM, flash memory, etc.), or some combination of the two, accessible by the processing unit(s). The memory 1320, 1325 stores software 1380 implementing one or more innovations described herein, in the form of computer-executable instructions suitable for execution by the processing unit(s).

A computing system may have additional features. For example, the computing environment 1300 includes storage 1340, one or more input devices 1350, one or more output devices 1360, and one or more communication connections 1370. An interconnection mechanism (not shown) such as a bus, controller, or network interconnects the components of the computing environment 1300. Typically, operating system software (not shown) provides an operating environment for other software executing in the computing environment 1300, and coordinates activities of the components of the computing environment 1300.

The tangible storage 1340 may be removable or non-removable, and includes magnetic disks, magnetic tapes or cassettes, CD-ROMs, DVDs, or any other medium which can be used to store information in a non-transitory way and which can be accessed within the computing environment 1300. The storage 1340 stores instructions for the software 1380 implementing one or more innovations described herein.

The input device(s) 1350 may be a touch input device such as a keyboard, mouse, pen, or trackball, a voice input device, a scanning device, or another device that provides input to the computing environment 1300. The output device(s) 1360 may be a display, printer, speaker, CD-writer, or another device that provides output from the computing environment 1300.

The communication connection(s) 1370 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

Any of the disclosed methods can be implemented as computer-executable instructions or a computer program product stored on one or more computer-readable storage media (e.g., non-transitory computer-readable media, such as one or more optical media discs such as DVD or CD, volatile memory components (such as DRAM or SRAM), or nonvolatile memory components (such as flash memory or hard drives)) and executed on a computer (e.g., any commercially available computer, including smart phones or other mobile devices that include computing hardware). By way of example and with reference to FIG. 13, computer-readable storage media include memory 1320, memory 1325, and/or storage 1340. The term computer-readable storage media does not include signals and carrier waves. In addition, the term computer-readable storage media does not include communication connections (e.g., 1370).

Any of the computer-executable instructions for implementing the disclosed techniques as well as any data created and used during implementation of the disclosed embodiments can be stored on one or more computer-readable storage media (e.g., non-transitory computer-readable media). The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A system for text translation, the system comprising:
a data store configured to store a database comprising a plurality of content items, the plurality of content items each comprising text; and
a processor configured to:
generate a base knowledge level of terminology;
generate and transmit a translated content item having translated text, wherein the translated content item is based at least in part on a selected one of the plurality of content items and wherein the translated text is generated based at least in part on the base knowledge level;
generate and transmit a query to verify an understanding of the translated content item; and
update the base knowledge level of terminology in the database based on a response to the query received by the processor.

2. The system of claim 1, wherein the processor is further configured to:
generate a plurality of actions, wherein the plurality of actions is based at least in part on the selected one of the plurality of content items.

3. The system of claim 1, wherein generating the plurality of actions comprises determining a problem, wherein the problem is based at least in part on the select one of the plurality of content items.

4. The system of claim 2, wherein the processor is further configured to:
receive verification of an understanding of the plurality of actions.

5. The system of claim 2, wherein the processor is further configured to:
receive a selection of at least one of the plurality of actions; and
generate and transmit a treatment based at least in part on the selection.

6. A method of text translation and action generation, the method comprising:
accessing a profile stored in a database, wherein the profile comprises a base knowledge level of terminology;
determining an understanding of a translated content item having translated text, the determination based at least on a response received regarding the translated text;
updating the base knowledge of terminology in the profile based at least on the response received regarding the translated text; and
determining a plurality of actions for a diagnosis based on the translated content item.

7. The method of claim 6, further comprising translating text in the plurality of actions.

8. The method of claim 7, further comprising determining an understanding of the plurality of actions based at least on a response received based at least in part on a translated text in the plurality of actions.

9. The method of claim 8, further comprising updating the base knowledge of terminology in the profile based at least on the response received regarding the translated text in the plurality of actions.

10. A non-transitory computer readable storage medium storing computer-executable instructions that when executed perform a method of text translation, the method comprising:
receiving, by a server, a content item comprising text;
generating and transmitting, by the server, a translated content item, wherein the translated content item comprises translated text in the content item;
verifying, by the server, an understanding of the translated content item; and
generating and transmitting, by the server, a plurality of actions based on a diagnosis of a condition identified from the content item.

11. The non-transitory computer readable storage medium of claim 10, further comprising verifying an understanding of the plurality of actions.
